(19) United States
(12) Patent Application Publication
(10) Pub. No.: US 2013/0274143 A1
(43) Pub. Date: Oct. 17, 2013

(54) USE OF DETECTOR RESPONSE CURVES TO OPTIMIZE SETTINGS FOR MASS SPECTROMETRY

(71) Applicants: Vincent A. Emanuele, II, Atlanta, GA (US); Brian M. Gurbaxani, Atlanta, GA (US)

(72) Inventors: Vincent A. Emanuele, II, Atlanta, GA (US); Brian M. Gurbaxani, Atlanta, GA (US)

(73) Assignees: The Government of the United States of America as represented by the Health and Human Services, Cent, Atlanta, GA (US); The Government of the USA as represented by the

(21) Appl. No.: 13/575,317

(22) PCT Filed: Oct. 7, 2011
(86) PCT No.: PCT/US11/55376

§ 371 Date: Jul. 26, 2012

Related U.S. Application Data
(60) Provisional application No. 61/390,910, filed on Oct. 7, 2010.

(51) Int. Cl. G01N 33/68

(52) U.S. Cl. CPC G01N 33/6851
USPC 506/12; 702/19

(57) ABSTRACT

Processes for identifying optimal mass spectrometer settings to produce the greatest confidence in sample constituent detection are provided. Data obtained on a mass spectrometer are analyzed by a quadratic variance function which accurately represents intensity variation as a variation of peak intensity. This function is then used to identify intensities that possess a minimum coefficient of variation that is useful for identifying optimal mass spectrometer settings. Inventive processes involve using a general purpose computer to identify optimal mass spectrometer settings for use in biomarker analyses, for optimizing peak detection and biomarker identification in a biological sample. The inventive processes provide for improved methods of identifying new biomarkers as well as screening subjects for the presence or absence of disease or biological condition.

### CROSS REFERENCE TO RELATED APPLICATIONS

This application claims priority of PCT Application No. PCT/US2011/055376 filed Oct. 7, 2011, the entire contents of which are incorporated herein by reference.

### GOVERNMENT INTEREST

The invention described herein may be manufactured, used, and licensed by or for the United States Government.

### FIELD OF THE INVENTION

The invention relates generally to mass spectrometry, and in particular to methods for surface enhanced laser desorption/ionization time-of-flight mass spectrometry (SELDI) signal preprocessing for improved relevant peak detection and reproducibility.

### BACKGROUND OF THE INVENTION

Surface enhanced laser desorption/ionization (SELDI) time-of-flight mass spectrometry is a useful technology for high throughput proteomics. While SELDI is user friendly compared to other mass spectrometry techniques, the reproducibility of peak detection has known limitations. SELDI and matrix assisted laser desorption/ionization (MALDI) mass spectrometry are technologies used to search for molecular targets that could be used for the early detection of diseases such as cervical cancer. This process is generally referred to as biomarker discovery. One critical step of this process is the optimization of experiment and machine settings to ensure the best possible reproducibility of results, as measured by the coefficient of variation (CV). The cost of this procedure is considerable man hours spent optimizing the machine, opportunity cost, materials used, and spent biological samples used in the optimization process. The reproducibility of peaks in SELDI mass spectrometry has been problematic. This has led to several important research articles studying experimental pre-analytic and analytic factors affecting reproducibility (1-4). Recently, several studies have been performed studying post-analytic factors of reproducibility, namely, the preprocessing of the data (5-8). These studies suggest that the choice of prior preprocessing algorithms leads to significantly different results with respect to the quality of the peaks found in the data.

Preprocessing methods could be improved by incorporating characteristics of the measurement process. Thus, there exists a need for an improved method of signal preprocessing for improved reproducibility in mass spectrometry platforms such as SELDI and MALDI.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A process is provided that is useful for identification of optimum mass spectrometer instrument settings, for the identification of biomarkers, and for improving relevant peak detection that is rapid, reproducible, and robust. A process includes subjecting a sample to SELDI or MALDI mass spectrometry to produce a first mass data set, performing a fit of at least a portion of the first data set to a quadratic variance model to obtain a first quadratic variance function, obtaining a first coefficient of variation function from the first quadratic variance function, and identifying a first objective function in said coefficient of variation function. By repeating the process using the same sample set but by varying one or more instrument settings, one then is capable of determining a minimum of the first objective function and a second objective function, wherein the instrument detection parameters used at the minimum represent optimized instrument detection parameters. The process is repeated any number of times at any desired number of different instrument settings. The mass spectrometer is then adjustable to the identified optimum instrument settings for subsequent or simultaneous use for test samples or regions. Various regions of the data set(s) are operable to identify optimum instrument settings such as data between sample peaks within the data set, control background samples, or combinations thereof. The resulting quadratic variance functions are optionally proteinaceous.

Also provided are processes for performing SELDI or MALDI comprising mass spectrometry including subjecting a sample to SELDI or MALDI mass spectrometry, obtaining a mass spectrum comprising detection data from the sample, subjecting the data to quadratic variance preprocessing to create preprocessed data, and generating a preprocessed mass spectrum from the step of subjecting.

The processes are optionally used for identifying the presence or absence of a biomarker in a test sample. The preprocessed mass spectrum or preprocessed data set are then used for reliable peak detection where the presence or absence of peaks identifies the presence or absence of a biomarker in the sample. It is appreciated that a biomarker is any identifiable biomarker including protein, lipid, molecules typically with a molecular weight in excess of 1 kD, or other known biomarker type.

### BRIEF DESCRIPTION OF THE DRAWINGS

FIG. 1 illustrates quadratic variance functions that fit SELDI data using differing buffer samples;

FIG. 2 is a plot of variance against mean intensity where the gray circles indicate mean/variance points estimated from regions in between peaks in the spectra; the solid black line is the best fit quadratic variance function; and while the dashed black lines indicate plus/minus one standard error;

FIG. 3 illustrates the number of predicted peaks at the 80% or more level found using LibSELDI and Ciphergen Express as shown by box-plots with the y-axis indicating number of peaks predicted in a QC spectrum;

FIG. 4 illustrates mean peak heights and peak height variances of peaks where the circles indicate the mean/variance pairs from non-peak regions used to estimate the model; the dark gray plus symbols correspond to peaks occurring in at least 80% of QC spectra; while the light gray plus symbols indicate peaks occurring in 50% to 80% of QC spectra; the dashed and dotted lines indicate one and two standard errors from the mean, respectively;

FIG. 5 illustrates one experimental SELDI result demonstrating mean peak heights and peak height variances for very large mean height values are not consistent with the quadratic variance model for intensities greater than 12,000 ion counts;

FIG. 6 illustrates that observed CV% values of peaks are consistent with the quadratic variance model for peak intensities between 3,000 and 12,000 ion counts;

FIG. 7 is a flow diagram illustrating one embodiment of a process for identifying optimal experimental conditions such as instrument settings or sample preparation; and

FIG. 8 is a flow diagram illustrating one embodiment of a process for generating preprocessed data.

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes are described as an order of individual steps or using specific materials, it is appreciated that described steps or materials may be interchangeable such that the description of the invention includes multiple parts or steps arranged in many ways as is readily appreciated by one of skill in the art.

By default machine settings, a SELDI spectrum is the result of pooling/summing numerous single-shot spectra. Sköld et. al. studied the acquisition of single shot spectra and proposed a statistical framework for pooling the single shot spectra (10). They introduced an expectation-maximization algorithm for combining the spectra that results in improved peak heights in the pooled spectrum. Malyarenko et. al. (11) introduced a charge-decay model for the baseline in a SELDI spectrum and used time-series methods for the common preprocessing tasks. The inventors of the processes described herein and their equivalents identify a quadratic variance model for the response of a detector used for MALDI or SELDI, which optionally leads to preprocessing methods showing improved performance as described herein and additionally at (12).

The present invention has utility as a method for identifying optimum mass spectrometer detector, laser, pressure, or other setting parameter for improved detection or confidence in detected peaks in a test mass spectrum. The invention further provides unique preprocessing of mass spectrometry spectra generated by SELDI or MALDI methods that provide improved reproducibility and confidence in peak detection. While the description is primarily directed to data generated by SELDI mass spectrometry, the processes are equally applicable to other mass spectrometry platforms such as MALDI, among others known in the art.

A quadratic variance model is provided that successfully explains the variation in SELDI spectra generated from samples such that reproducibility is improved. The detector response curve idea can be used to optimize the coefficient of variation (CV) with the following advantages over conventional methods: 1) no need to use biological samples to determine machine settings and model parameters to apply to actual data; 2) fewer materials used in the process; 3) improved CV and thus more reproducible results; 4) fewer man hours required to find good machine settings; and 5) optional full-automation of the process of optimizing CV. The inventive algorithms for peak detection based on the quadratic variance model are used in some embodiments to analyze SELDI spectra from multiple aliquots of a single pooled cervical mucous sample used as quality control (QC) for SELDI. These inventive results are optionally compared to peak detection with the vendor supplied Ciphergen software (13) and found favorable. As each spectrum is a replicate of one sample, all should have the same number of proteins and thus yield reproducible peaks. From this point of view, increasing the number of peaks found consistently indicates improved performance of a preprocessing technique.

The following abbreviations are used throughout the specification: Surface-enhanced laser desorption/ionization time-of-flight mass spectrometry (SELDI-TOF MS or SELDI), Matrix-assisted laser desorption/ionization (MALDI), quadratic variance function (QVF), mean intensity (μ), variance (V), kiloDalton (kDa), microliter (μL), liquid chromatography/tandem mass spectrometry (LC-MS/MS).

Some embodiments of an inventive process include subjecting a first sample to SELDI or MALDI mass spectrometry and obtaining a mass data and/or a mass spectrum from the first sample. A fit of at least a portion of said mass spectrum to a quadratic variance model is performed to obtain a quadratic variance function (QVF). A process may also include converting the parameters of the QVF to obtain a coefficient of variation (CV) for each peak. The QVF can also be converted to a coefficient of variation function. An objective function of the coefficient of variation function is used to calculate a performance metric that represents the utility of the instrument detection parameters used. Then the optimal settings can be selected by choosing the parameters that minimize the objective function. Examples of useful objective functions/performance metrics are the maximum CV in a specified input intensity interval (a minimax risk approach), the area under the CV curve in a specified interval normalized by the length of the interval (an average risk approach), and the asymptotic “large” signal value of the CV function. Analyzing the coefficient of variation function or the objective function then allows for identifying an optimal machine parameter or set of parameters.

As used herein, the term “sample” is defined as a sample obtained from a biological organism, a tissue, cell, cell culture medium, or any medium suitable for mimicking biological conditions, or from the environment. Non-limiting examples include, saliva, gingival secretions, cerebrospinal fluid, gastrointestinal fluid, mucous, urogenital secretions, synovial fluid, cerebrospinal fluid, blood, serum, plasma, urine, cystic fluid, lymph fluid, ascites, pleural effusion, interstitial fluid, intracellular fluid, ocular fluids, seminal fluid, mammary secretions, vitreal fluid, nasal secretions, water, air, gas, powder, soil, biological waste, feces, cell culture media, cytoplasm, cell releasate, cell lysate, buffers, or any other fluid or solid media. A sample is optionally a buffer alone, water alone, or other non-protein containing material. A sample is optionally pooled from a plurality of subjects.

A “subject” as used herein illustratively includes any organism capable of producing a proteinaceous sample. A subject is illustratively a human, non-human primate, horse, goat, cow, sheep, pig, dog, cat, rodent, insect, or cell.

A sample is subjected to analysis by mass spectrometry. Mass spectrometry is optionally any spectrometry that requires desorption of a sample, or portion thereof, from a surface or from a fluidic sample. Illustratively, mass spectrometry is performed by laser desorbtion. Illustrative examples of mass spectrometry that use laser desorbtion include MALDI or SELDI. Methods of MALDI and SELDI are well known in the art. Illustratively, methods of SELDI can be found at Emanuele, V. A. and Gurbaxani, B. M., BMC Bioinformatics, 2010; 11:512. Methods of subjecting a sample to MALDI are illustratively found in Gould, W R, et al., J Biol Chem, 2004; 279(4):2383-93 and references cited therein.

A mass data set and, optionally a representative mass spectrum, is optionally obtained from the first sample. A mass data set represents the relative abundance of material in a sample as defined by intensity as a function mass/charge ratio. A mass data set is illustratively presented graphically (e.g. mass spectrum), or as a collection of data points. The mass data set is fit to a quadratic equation as follows:

V(μ)=v₀+v₁μ+v₂μ². (Eq.1)

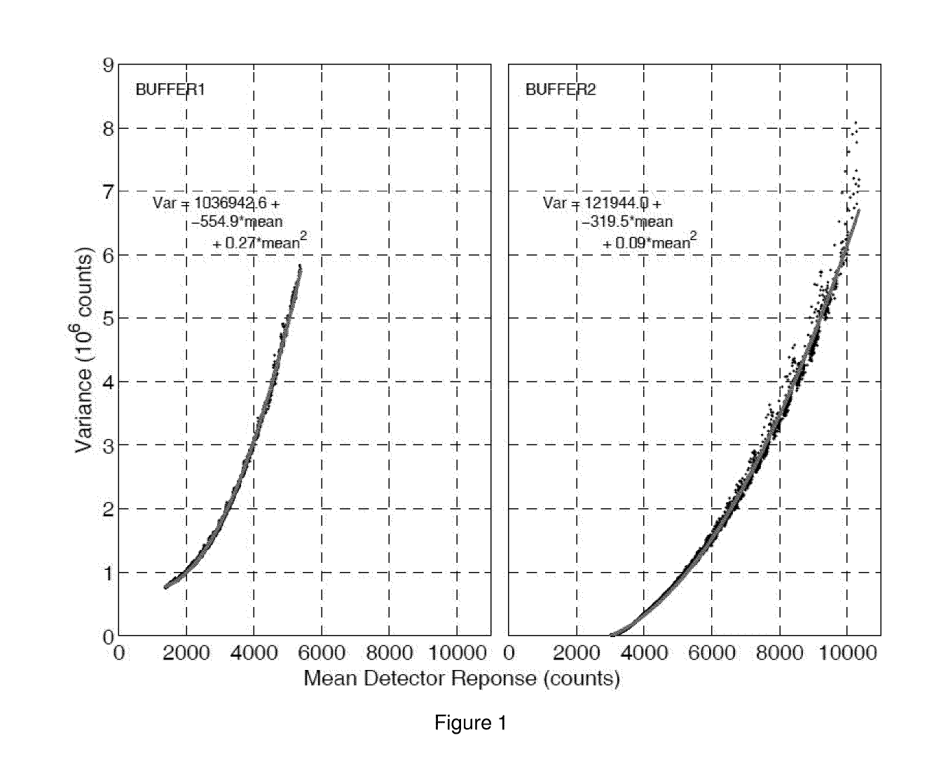

with μ being the mean of the intensity at a particular mass/charge ratio (X), V(μ) the variance, and v₀, v₁, v₂ constants, some of which may be zero. The fit of the mass spectrum to Equation 1 provides values for the constants v₀, v₁, and v₂. It is observed that different experimental conditions provide different quadratic variance functions as illustrated in FIG. 1 for background spectra from two different buffer conditions. Different quadratic variance functions are also observed for differing instrument settings providing a basis for instrument optimization processes.

The obtained quadratic variance function is then optionally used to obtain a coefficient of variation function as defined by:

$$\begin{matrix}CV \% = 100 \cdot \frac{σ}{\vert μ \vert} \\ = 100 \cdot \sqrt{\frac{V ( μ )}{{μ}^{2}}} \\ = 100 \cdot \sqrt{{υ}_{0} {μ}^{- 2} + {υ}_{1} {μ}^{- 1} + {υ}_{2}}\end{matrix} \qquad ( Eq . 2 )$$

It is recognized that Equation 2 has a plurality of objective functions each of which are be readily identified by methods known in the art. For example, varying machine settings provide the minimum area under the CV curve in a specified interval normalized by the length of the interval (an average risk approach). This can then be used to identify mass spectrometer settings that produce optimal results.

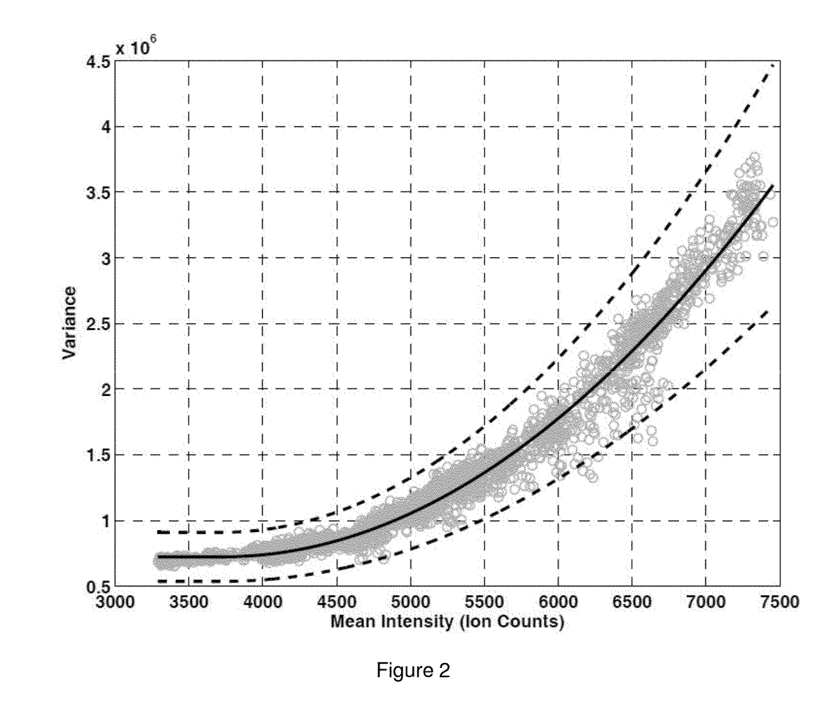

FIG. 2 illustrates observed variance as a function of mean intensity for the gaps between peaks in QC spectra (circles) obtained from pooled cervical samples, and the quadratic variance function fit (using Equation 1) to the same (solid line), plus or minus 1 standard error (dashed lines). Very few points fall outside of 1 standard error. This confirms that the area interspersed between peaks follow the quadratic variance model.

In some embodiments, a sample is a proteinaceous sample. As an illustration, a proteinaceous sample produces one or more mass spectra that are used to obtain a quadratic variance function with a variance that is constant for a peak with a mean intensity at or below a lower threshold value. A quadratic variance function optionally has a quadratic dependence of variance as a function of mean intensity above the lower threshold value. In some embodiments, a quadratic variance function has an upper threshold value at or above which the variance is constant as a function of mean intensity. In some embodiments, a lower threshold value is 3,700 ion counts. An upper threshold value is optionally 12,000 ion counts. A lower threshold value and an upper threshold value are appreciated to vary depending on the instrument used, instrument settings, sample type, matrix type, or background type. It is further appreciated that one of skill in the art can readily determine the value of a lower threshold value and an upper threshold value by mathematical analysis of the quadratic variance function. Illustratively, a threshold value (either lower or upper) is identified by taking the first derivative of the quadratic variance function, and noting when that derivative becomes a constant (equal to zero at a lower threshold or some positive constant at an upper threshold).

In some embodiments, a plurality of mass data sets are obtained from a single sample, or from a plurality of samples. The plurality of mass data sets are optionally obtained at different mass spectrometer settings. Illustratively, an operator may alter or otherwise adjust parameters including laser intensity, detector sensitivity, ion mode, extraction delay, flight tube length, pressure, temperature, laboratory protocols that affect the preparation of the sample on the chip, other parameter, or combinations thereof.

A process optionally further includes adjusting mass spectrometer detection settings to said optimal detection parameters. Adjusting mass spectrometer settings is optionally performed by a user or automatically on the instrument itself. Illustratively, a user identifies the objective function minimum from one or a plurality of coefficient of variation functions optionally obtained at varying mass spectrometer settings. The mass spectrometer settings used at the objective function minimum represents optimal instrument detection parameters for the plate or sample conditions.

In some embodiments, a mass spectrometer is programmed to automatically identify a minimum in the objective function measure of the coefficient of variation function obtained from one or a plurality of mass data sets. As an example, a first sample, or a plurality of samples are subjected to mass spectrometry analysis. For each sample, a quadratic variance function is obtained by a fit of at least a portion of the mass data set generated. The fit is optionally performed on a general purpose computer that is separate from or associated with the mass spectrometer. The fit is then used to obtain one or a plurality of coefficient of variation functions that each may be evaluated for merit via the chosen objective functional. The lowest minimum of the objective function of one or plurality of coefficient of variation functions represents the optimal instrument detection parameters. This is readily identified by the program of the instrument. The instrument detection parameters are then automatically adjusted by the instrument for subsequent subjecting of the first sample, a second sample, or one or more other samples to mass spectrometry analysis.

In some embodiments, a process includes subjecting data generated in a mass spectrometer to quadratic variance preprocessing to create preprocessed data. The preprocessed data are then used for reliable peak detection, to generate a mass spectrum from the preprocessed data, or for other purposes recognized in the art. The process of subjecting data to quadratic variance preprocessing are essentially as described by Emanuele, V, and Gurbaxani, B., BMC Bioinformatics, 2010; 11:512. One or more mass spectra generated on a mass spectrometer as the result of SELDI are collected.

The inventive processes are illustrated by application to repeat testing of a pooled cervical mucus sample using a Protein Biology System II-c mass spectrometer. The invention uses a set of MATLAB® scripts (The MathWorks, Inc., Natick, Mass.) for preprocessing SELDI spectra termed by the inventors as LibSELDI. Spectra from blank, control, or test samples generated are preprocessed with LibSELDI, based on a quadratic variance model, and optionally compared to the other peak detection systems, illustratively, Ciphergen Express (Bio-Rad Laboratories, Inc., Hercules, Calif. Peak predictions from both algorithms are gathered into homogenous clusters and peak prevalences and CV % of peak heights are calculated and compared with predictions from the quadratic variance model.

In one test embodiment, the inventive quadratic variance based algorithm finds 84 peaks occurring in at least 80% of the spectra from pooled cervical mucus sample while Ciphergen finds only 18 such peaks (FIG. 2). The predictions of the quadratic variance model match the observed peak height variances and peak height CV %. The inventive pre-processing approach (synonymously referred to herein as “LibSELDI”) based on the quadratic variance model finds four times as many reproducible peaks in the pooled cervical mucous samples as Ciphergen Express. Also, the model successfully assesses the CV % likely to be observed by making measurements of blank spectra giving rise to new ways to optimize machine parameters. Thus, the inventive quadratic variance model based approach detects peaks more reproducibly thereby increasing the utility of SELDI.

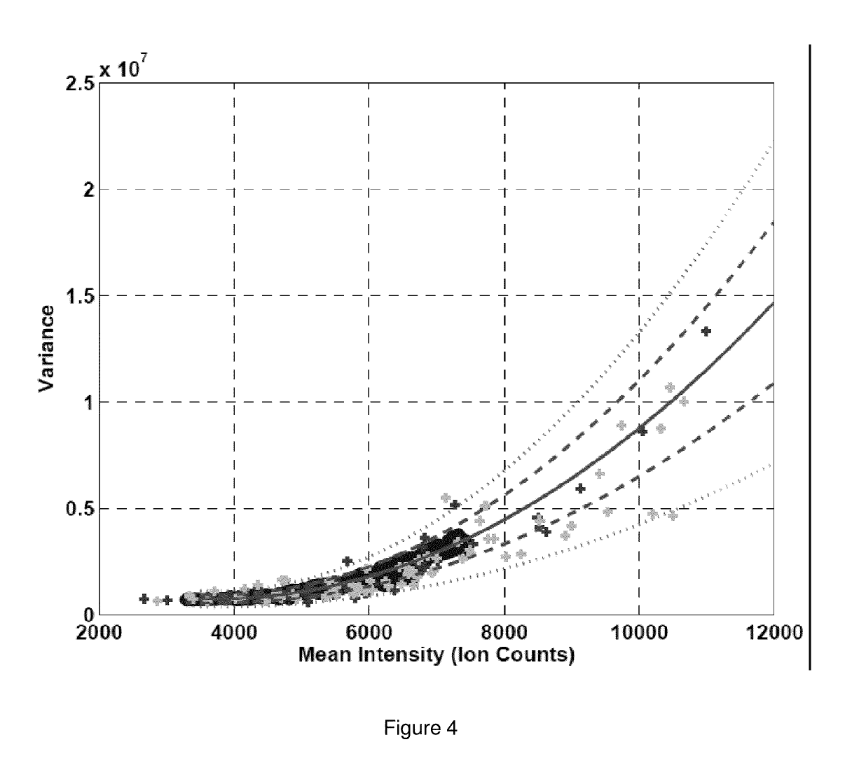

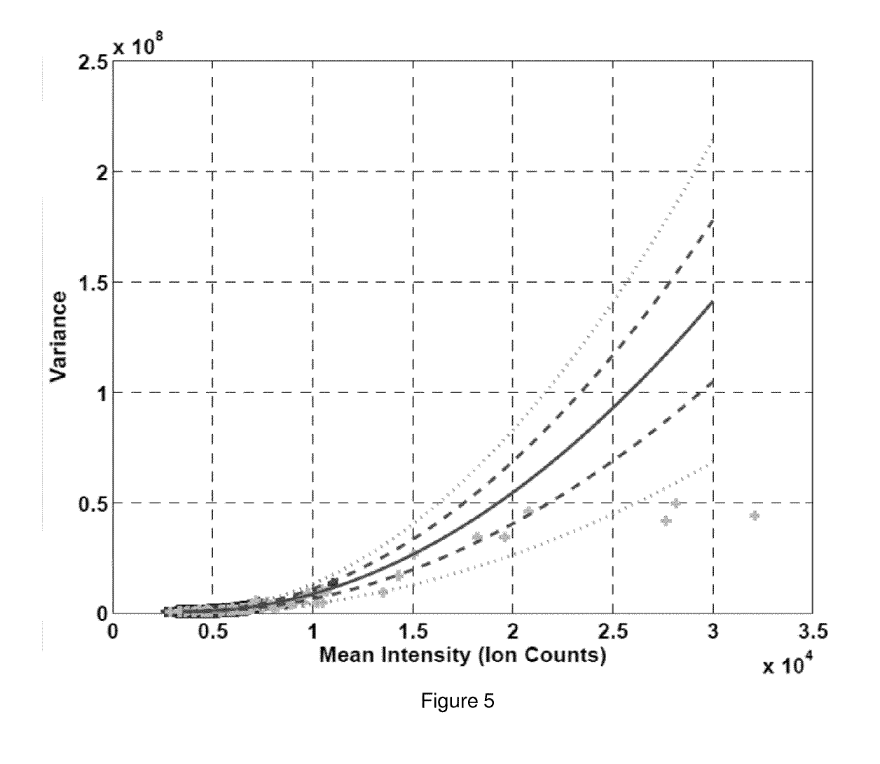

Reproducible peaks show peak height variances that are consistent with the quadratic variance model. This provides an indication of how the noise varies with proteins with different abundances. Analysis are optionally restricted to peaks appearing in at least 50% of the spectra (guaranteeing at least n=16 for sample means and variances). This is illustrated for the range of intensity values encompassing most of the peaks in FIG. 4. For the few cases with peaks of very high mean intensity (such as those lying above an upper threshold value e.g. >12,000 ions counts for SELDI, which may vary for a different instrument such as a MALDI instrument, occurring in the spectra, the quadratic function becomes substantially linear. This is illustrated in FIG. 5.

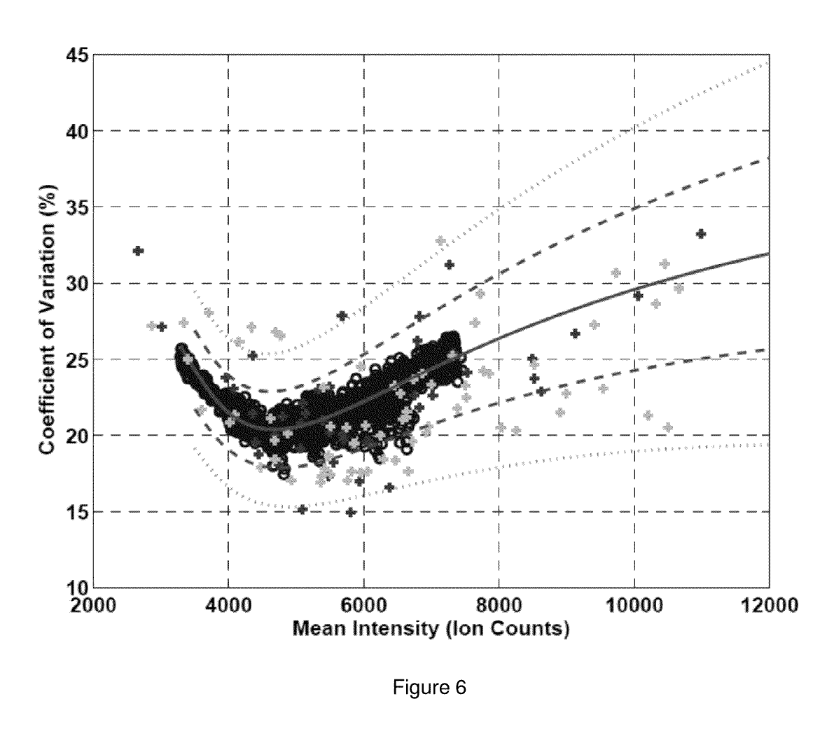

The CV % of peak height intensity for the reproducible peaks agree with the quadratic variance model, showing which ranges of abundances give the best and worst CV % for these machine settings, as illustrated in FIG. 6. Similar to FIG. 5, the model becomes constant for peaks at very high mean intensity (e.g. above 12,000 ion counts for SELDI in this embodiment), which are a small minority of observations. However, the predictions are still bounded below the large μCV approximation predicted by the model in Eq. (3).

Using the LibSELDI algorithm for pre-processing based on the quadratic variance model to explain the variation in SELDI signal detection results in significantly improved peak detection and reproducibility of peak detection compared to the Ciphergen algorithm. The affinity for finding peaks occurring in more than 80% of the spectra is impressive—finding more than four times as many as Ciphergen (84 peaks versus 18). The higher number of peaks is consistent with direct measures on the same sample using, 2-D and 1D LC-MS/MS gel, which despite limited sensitivity, is able to detect 49 proteins in the mass range of 8.6-30 kDa (15). Several other studies doing proteomic analysis on a similar sample type, cervicovaginal fluid, have also shown it to be a complex sample with total number of proteins ranging from 59-685 (17-21).

The protein estimates/peaks found by the model have mean peak heights, variances, and CVs that are consistent with what is predicted. Thus, in simple terms, the quadratic variance function estimate predicts peak reproducibility as a function of intensity in advance of an experimental run optionally using “blank” regions of the spectra (between visible peaks), buffer alone, or modeled spectral data to derive parameters for the algorithm. This allows the algorithm to be adjusted for changing noise/background characteristics encountered with each set of experimental conditions. This also allows for identification of optimal instrument settings with minimized CV objective function optionally based on blank spectra prior to running samples.

In some embodiments, using proteinaceous samples as typically obtained from a biological sample, the quadratic variance model of measurement for SELDI shows a constant variance for mean intensities below 3,700 ion counts, quadratic between 3,700 and 12,000 ion counts, and transitioning to non-quadratic variance for very high intensities above 12,000 ion counts. The constant variance is optionally determined by calculating the first derivative at each portion of the curve. When the first derivative is zero or constant, a constant variance is identified at that point in the curve. Fortunately, most peak heights from exemplary pooled mucous QC samples are observed in the quadratic variance region.

The inventive algorithm is particularly advantageous in analyzing or identifying proteins, peptides, or other compositions with a molecular mass near 2.5 kDa, optionally anywhere from 1 kDa to 30 kDa, where the baseline hits a maximum due to non-linearities introduced by the detector saturating.

The use of the detector response curve (i.e. the value of the objective function as a function of instrument setting, illustratively in the case of SELDI) and its link to the coefficient-of-variation (CV) has many potential commercial applications. This invention is operative to design a MALDI/SELDI mass spectrometer that automatically optimizes itself before a biomarker discovery experiment (or any other experiment using this technology). This invention is also operative to use the detector response curve as part of a quality control (QC) technique. For this application, experimental data is compared on a computer to the typical measurements expected from the detector response curve and suspicious data can be automatically flagged for further inspection. This increases the reliability of the data coming from these instruments. Another potential use of the detector response curve is to tune the machine to pre-specified protein concentrations. For example, machine settings are set so that low, medium, or high intensity proteins show the best CV. This is useful in situations where one knows in advance the characteristics of the molecular target being searching for. The idea of a detector response curve is useful to a manufacturer of electron-multiplier detectors for MALDI/SELDI to assess which detector designs are superior for biomarker discovery studies.

### EXAMPLES

The present invention is further detailed in the following examples that are not intended to limit the scope of the claimed invention and instead provide specific working embodiments.

### Example 1

### Sample Collection and Processing

Cervical mucous is collected from women enrolled as part of an ongoing study of cervical neoplasia (14). At the time of colposcopy, two Weck-Cel® sponges (Xomed Surgical Products, Jacksonville, Fla.) are placed, one at a time, into the cervical os to absorb cervical secretions (15). The wicks are immediately placed on dry ice and stored at −80° C. until processed. Preparation of the pooled quality control (QC) sample is described (15). Briefly, 40 Week-Cel® sponges with no visual blood contamination from 25 randomly selected subjects are extracted using M-PER® buffer (Thermo Fisher Scientific, Rockford, Ill.) containing 1× protease inhibitor (Roche, Indianapolis, Ind.). The 40 extracts are combined, aliquoted and stored at −80 ° C. until assayed. Total protein content is measured using the Coomasie Plus™ kit (Thermo Fisher Scientific) as per the manufacturer's protocol.

### Example 2

### SELDI-TOF Mass Spectrometry

A Protein Biological System II-c™ mass spectrometer, with Protein Chip software (version 3.2) (Ciphergen Biosystems, Fremont, Calif.) is used to perform SELDI-TOF MS. The mass calibration standard (All-in-one protein standard, Ciphergen) spotted on the NP-20 (normal phase) chip surface (Ciphergen) is run weekly, following manufacturer's instructions. Pooled cervical mucous is spotted on chips intermittently as part of a QC step in the experiment design. Protein chip surface preparation, sample application and application of matrix are performed using the Biomek® 2000 laboratory automation workstation (Beckman Coulter Inc., Fullerton, Calif.) according to the manufacturer's (Ciphergen) instructions.

The CM10 chips evaluated are incubated with the sample for 1 h at room temperature (24° C.±2) and washed three times at 5 min intervals with the CM10 low stringency binding buffer, followed by a final wash with ddH2O. In the case of NP-20 arrays, the surface is prepared with 3 μl ddH2O, and ddH2O is used for all washing steps. Chips are air-dried 30 min prior to the application of sinnapinic acid (SPA) matrix. The chips are analyzed on the SELDI-TOF instrument within 4 h of application of the matrix.

Buffer-only spectra were generated by interspersing buffer only samples with protein samples from subjects (e.g. serum samples) and with pooled subject samples on the same chip. The buffer-only samples were spotted with wash buffer that was either PBS (phosphate buffered saline with various concentrations of phosphate and NaCl) based or acetonitrile+TFA (triflouroacetic acid) based, as manufacturer recommended per chip type. These buffer only samples were processed with the same washing steps as the subject samples, and then SPA matrix was applied to all spots.

The instrument settings are determined separately for the low mass and high mass range of the protein profile. Data collection is set to 150 kDa optimized for m/z between 3-30 kDa for the low mass range and 30-100 kDa for the high mass range. For the low mass range, the laser intensities are set at 185 with a detector sensitivity of 8 and number of shots averaged at 180 per spot for each sample. Two warming shots are fired at each position with the selected laser intensity +10. These are not included in the data collection. Data collection from start to finish took 2 weeks and included a total of 31 spectra.

### Example 3

### Detector Response Curve Estimation

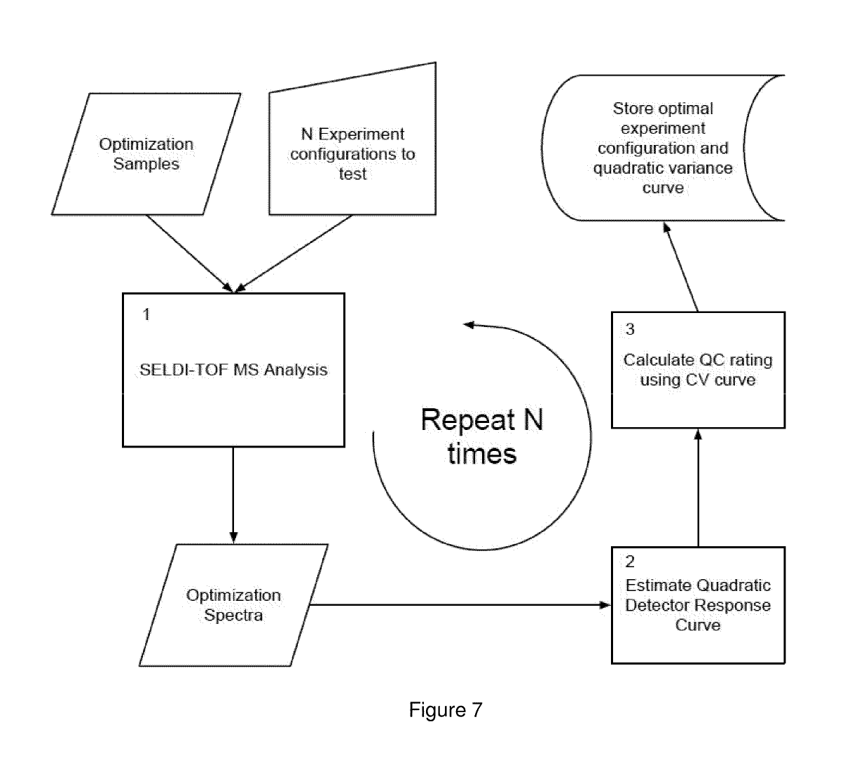

The quadratic variance model is used to characterize the measurement of the intensity values registered at the ion detector in response to a wide range of signal levels. The variance of the detector response is quadratic with respect to the mean intensity level as observed in a repeated experiment. To show this, we used data taken from buffer, matrix-only spectra containing no biological signal or protein content as described (12). Extending this idea to our current study, we estimated the detector response curve by using hand selected regions where peaks are visibly absent in all of the QC spectra. An illustrative process is presented in FIG. 7. A sample is subjected to SELDI analysis as in Example 2 (block 1). As represented in block 2 of FIG. 7, the quadratic variance model implies that the mean intensity μ of repeated measurements and corresponding variance V(μ) have the relationship

V(μ)=v₀+v₁μ+v₂μ². (Eq.1)

with μ being the mean of X, V(μ) the variance, and v₀, v₁, v₂ constants, some of which may be zero. The variance V(μ) is best estimated for the range of intensities used to estimate the curve, but this extrapolates well to values outside this range.

The quadratic variance function for the detector response is used to predict how peak intensities will behave in the spectra of a repeated SELDI experiment. One subtle aspect of Eq. (1) is that it predicts what the CV of such measurements will be (represented as block 3 of FIG. 7),

$$\begin{matrix}CV \% = 100 \cdot \frac{σ}{\vert μ \vert} \\ = 100 \cdot \sqrt{\frac{V ( μ )}{{μ}^{2}}} \\ = 100 \cdot \sqrt{{υ}_{0} {μ}^{- 2} + {υ}_{1} {μ}^{- 1} + {υ}_{2}} \\ \approx 100 \cdot \sqrt{{υ}_{2}} ( μ large ) . ( Eq . 3 )\end{matrix} \qquad ( Eq . 2 )$$

Equation 3 merely states that when the mean signal intensity is large, the coefficient of variation is approximately constant since the other terms dependent on μ becoming negligible. Altogether, equations 1-3 provide intuition and are sufficient to make predictions about optimal instrument detection parameters for the same or other experimental runs. As an example, data between peaks is used for a determination of the values for Eq. 1. This provides simultaneous test data acquisition and allows determination of the v₀, v₁, and v₂ coefficients for the experimental conditions (sample, chip and instrument settings), and therefore the mean heights and variances, as well as the CV's, of peaks for the experiment. For very large peaks (e.g. high intensity >12,000), the CV % of peak heights is approximated by 100·\sqrt{v₂} as demonstrated in FIG. 7.

### Example 4

### Pre-Processing with LibSELDI

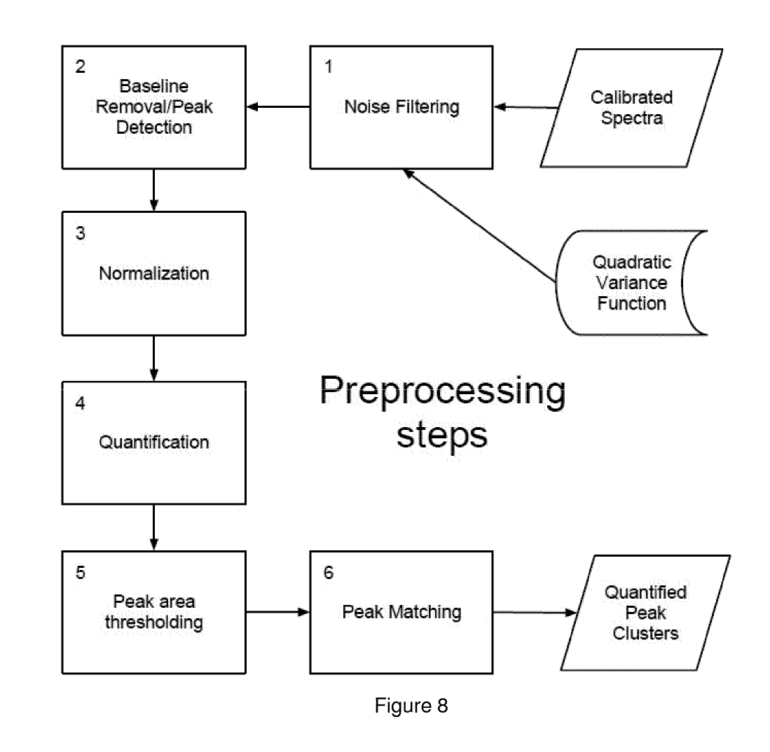

The LibSELDI preprocessing package is developed in MATLAB (The Mathworks, Natick, Mass.) and takes into account a quadratic variance form of the measurement error. The details of the algorithms used by LibSELDI are described by Emanuele, V. A. and Gurbaxani, B. M., BMC Bioinformatics. 2010; 11: 512. LibSELDI is used to process the data adhering to the following protocols: A single quadratic variance function (QVF) is estimated representing all 31 QC spectra; The QVF is estimated according to the procedure described in Example 4; Preprocessing is performed on each spectrum individually rather than the mean spectrum. A flowchart of the steps involved in preprocessing are illustrated in FIG. 8.

Multiple Spectra Considerations.

Rather than observe a single spectrum, the typical biomarker discovery approach is to generate at least one spectrum for each of n samples from an approximately homogeneous population. For example, the homogeneous population of Example 2 is studies. As the samples are run on the same SELDI machine with the same operating conditions, we have

X₁(t), . . . , Xₙ(t) ∝ NEF−QVF (V(μ(t))). (Eq. 4)

The X₁, . . . Xₙ represents the optimization spectra for a single experiment/machine setup. A second, and optionally plurality of data sets are obtained under different instrument settings and the process is repeated.

The assumption that all n patients have the same underlying μ(t) is equivalent to assuming that the underlying biological condition being observed in each patient is approximately the same. Thus, underlying commonality μ(t) related to the biology of their condition expressed through the SELDI signal is estimated. Some of the effects of the QVF are mitigated by optionally forming a mean spectrum (first introduced by 22).

$$\begin{matrix}{X}_{\cdot } ( t ) = \frac{1}{n} {\sum }_{k = 1}^{n} {X}_{k} ( t ) . \\ therefore & ( Eq . 5 ) \\ E \{ {X}_{\cdot } ( t ) \} = μ ( t ) & ( Eq . 6 ) \\ Var ( {X}_{\cdot } ( t ) ) = \frac{1}{n} V ( μ ( t ) ) . & ( Eq . 7 )\end{matrix}$$

Modified Antoniadis-Sapatinas Denoising.

For generation of a preprocessed mass spectra, the data obtained as in Example 2 are subjected to modified Antoniadis-Sapatinas denoising represented as block 1 of FIG. 8. μ(t) from the mean spectrum obtained by a fit of the means spectrum to Eq. 5 Since the Xₖ(t) are sampled on a discrete time grid (and thus X•), a vector notation is introduced.

x•=[X•(t₁), . . . , X•(tₘ)^{'}

μ=[μ(t₁), . . . , μ(tₘ)]^{'}. (Eq. 8)

or any estimate \hat{μ}(x•)of, μ we measure its fitness using the mean-squared-error (MSE).

MSE(\hat{μ}(x•),μ)=E{∥\hat{μ}(x•)−μ∥²}. (Eq. 9)

For denoising, we use the orthogonal discrete wavelet transform with respect to the Symmlet 8 basis. The transform is represented by an m x m orthogonal matrix W.

w=Wx•. (Eq. 10)

Where h is a length m vector with entries taking values between 0 and 1. Let H=diag(h) be the m×m matrix defined by placing the entries of h along the main diagonal, all other entries 0. The class of estimators for \hat{μ}(x•) take the form

$$\begin{matrix}\hat{μ} ( {x}_{\cdot } ) = {W}^{′} Hw \\ = {W}^{′} H W {x}_{\cdot } .\end{matrix} \qquad ( Eq . 11 )$$

This is the typical wavelet denoising scenario where each wavelet coefficient is left alone or shrunk towards zero according to some criterion, and is completely defined by the vector h. Antoniadis and Sapatinas showed that a good estimator for data from the NEF-QVF family is given by choosing:

$$\tilde{h} ( i ) = \frac{[ {w ( i )}^{2} - {\hat{σ}}^{2} ( i ) ] +}{{w ( i )}^{2}} , i = 1 , … , m \\ [ z ] += \{ \begin{matrix}z , & z \geq 0 \\ 0 , & z < 0.\end{matrix} \qquad ( Eq . 12 )$$

where the term \hat{σ}² is estimated as

$${\hat{σ}}^{2} = \frac{1}{1 + {υ}_{2}} ( W \cdot W ) V ( {x}_{\cdot } ) \text{:} \qquad ( Eq . 13 )$$

where V(x•) is the vector constructed by applying the QVF from (1) to each term of x•. (W·W) is the matrix whose i, j element is the square of the i, j element of W. The parameters v₀, v₁, v₂ in Eq. 1 are measured from the background regions, buffer only spectra, or prior test sample data as in Example 3.

An intuitive modification is made to Eq. 13 to obtain:

$${\tilde{σ}}^{2} = \frac{1}{1 + {υ}_{2}} ( W \cdot W ) {V}^{†} ( {x}_{\cdot } ) . \\ {V}^{†} ( {x}_{\cdot } ( i ) ) = max \{ V ( {x}_{\cdot } ( i ) ) , {υ}_{0} \} . \qquad ( Eq . 14 )$$

Thus, the modified Antoniadis and Sapatinas estimator \tilde{h} uses \hat{σ}² in Eq. 12 rather than \hat{σ}². The modification was introduced to account for cases when Eq. 13 may underestimate the noise when low amounts of observed signal are detected. Define

$$\tilde{h} = \frac{[ {w ( i )}^{2} - {\tilde{σ}}^{2} ( i ) ] +}{{w ( i )}^{2}} \\ \tilde{H} = diag ( \tilde{h} ) . \qquad ( Eq . 15 )$$

then, the modified Antoniadis-Sapatinas estimate of μ is defined as

\tilde{μ}=W^{'}\tilde{H}Wx•. (Eq. 16)

Peak Detection/Baseline Removal.

For peak detection and baseline removal the two preprocessing steps of baseline removal and peak detection typically performed separately are consolidated into a single step. These processes are represented in block 2 of FIG. 8. It is assumed that the underlying μ(t) shown in Eq. 6 is the superposition of protein ions, s(t), and energy-absorbing matrix ions, b(t) striking the detector. The distribution of the isotopes in the analyte of interest gives rise to a roughly Gaussian peak shape. Thus, it is proposed that

$$\begin{matrix}μ ( t ) = s ( t ) + b ( t ) & ( Eq . 17 ) \\ s ( t ) = {\sum }_{j} {a}_{j} {𝒢}_{3 {σ}_{j}} ( {t}_{j} , {σ}_{j} ) & ( Eq . 18 )\end{matrix}$$

where, G_{α}(tⱼ,σⱼ) denotes a Gaussian kernel function centered at tⱼ with standard deviation σⱼ and zero outside the interval [tⱼ−α, tⱼ+α].

Typically, s(t) is very sparse in the sense that it is mostly zero over the domain of the observed signal. Therefore, the local minima of the estimated baseline+noise signal \tilde{μ} are points that may be assumed to touch the baseline. From this point of view, once all the local minima in \tilde{μ} are detected, the baseline curve estimation reduces to an interpolation amongst these points. For this purpose, piecewise cubic Hermite interpolating polynomials (as performed in ref. 23) are excellent interpolation functions.

The minima and maxima in \tilde{μ} are found in one pass using the extrema function downloadable from MATLAB® central file exchange (finds all locations where the first derivative of \tilde{μ}=0). The maxima are the peaks in the mean spectrum potentially indicating proteins represented in the sample population of Example 2 while the minima correspond to samples from the baseline signal.

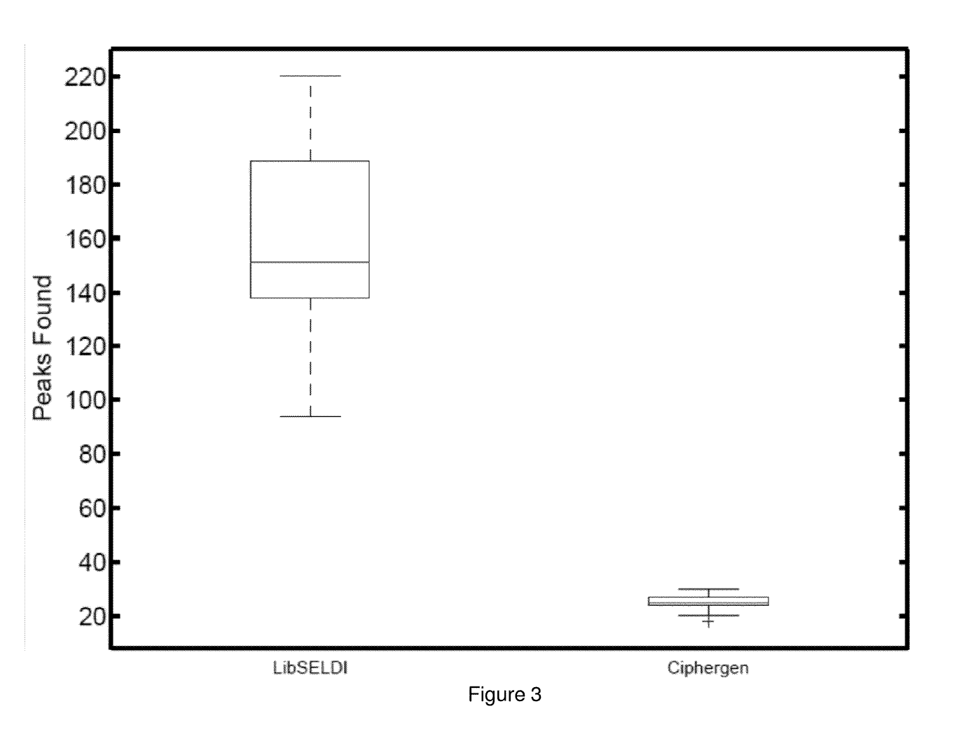

Normalization of block 3 of FIG. 3 is achieved by any standard normalization method known in the art. Illustratively, the normalization method is that of Meuleman et al., BMC Bioinformatics 2008; 9:88.

Each detected peak is quantified using peak area and a threshold is chosen based on the peak area measurement to generate the final prediction set as represented in blocks 4 and 5 of FIG. 8.

### Example 5

### Pre-Processing with Ciphergen

All SELDI spectra of Example 2 are processed using Ciphergen Express Client software (version 3.0). Pre-processing of the spectra is performed as previously described (16). Briefly, baseline correction, external calibration using protein standards, normalization using total ion current, and mass alignment are applied to all spectra. Peak detection is performed on this pre-processed data. Peaks from 2.5-30 kDa are detected by centroid mass, with first pass settings of signal to noise ratio (S/N)=5, valley depth=3, second pass settings of S/N=3 and valley depth=2, and a mass window of 0.3%.

### Example 6

### Peak Matching

When peak predictions are made in a repeated experiment, it is useful to group peaks from distinct spectra that are close enough in m/z value to be assumed to be generated from the same underlying analyte. This allows one to assess the reproducibility of a peak in terms of its prevalence (% of times it appears across spectra) and CV (of both peak m/z and peak intensity). This process is referred to as peak matching or peak clustering.

A fair comparison of reproducibility of peak predictions requires that the same peak matching algorithm be used for each method. Otherwise, one could not ascertain whether the core preprocessing algorithms (denoising, baseline removal, peak detection) or the peak matching algorithms contributed most to conclusions about the superiority of one preprocessing approach versus another. LibSELDI and Ciphergen use different peak matching techniques, with the Ciphergen approach being an unpublished, proprietary method. For this reason, LibSELDI's peak matching algorithm is used to assess prevalence and CV's for both preprocessing programs' peak predictions. Since the peak matching algorithm is completely independent of the methodology used in the core preprocessing steps of both Ciphergen and LibSELDI, there is no reason to believe it would give either algorithm an advantage in this comparison. The results are presented in FIG. 3 demonstrating improved reproducible peak detection by the LibSELDI process.

### Example 7

### Estimation of Parameters for Peak Clusters

For each peak in a peak cluster, the analyte mass is estimated using the detected peak m/z location of the smoothed, processed spectrum obtained as in Example 4 and is illustrated as block 6 in FIG. 7. The peak height is measured as the maximum intensity value observed in a window centered around the peak m/z value. The peak area is measured as the sum of intensity values observed in a window centered around the peak m/z value. The mean, variance, and CV of peak heights and peak areas are then calculated for each peak cluster. Note that, this is slightly different from measuring mean and variances from the peak-free regions. For the peak-free regions mean and variance of intensity are calculated for each fixed m/z value.

### Example 8

### Optimization of Detector Settings

Thirty buffer only samples are prepared on sample plates and combined with SPA matrix as in Example 2. The buffer only samples are subjected to ionization in a SELDI mass spectrometer as described in Example 2 with varying detector sensitivity settings ranging from 5 to 9. Ten different detector sensitivities are studied using three spot per sensitivity setting. The resulting data sets are used to generate mass spectra and for identification of a quadratic variance function representing the data set, produce a resulting coefficient of variation function, and are processed to obtain an objective function as in Example 3. The objective function used in these studies is an area under the coefficient of variation function analysis for intensities ranging from 4,000 to 6,000. The minimum value for area under the curve from the 10 different settings is then chosen. The detector settings producing the minimum objective function value represent optimal instrument detector sensitivity settings for the buffer/matrix samples.

The above studies are repeated by obtaining 10 spectra at each detector sensitivity setting but at varying laser intensity settings with laser intensity low values ranging from 175 to 245 and laser high values ranging from 185 to 255. The data set of each spectrum is then subjected to the same analyses procedures. A 10×10 matrix or area under the curve is obtained with the two varying instrument settings. The minimum value in the matrix establishes the optimum instrument settings (laser intensity/detector sensitivity) for the buffer and matrix combination.

The instrument is then adjusted to the identified optimum instrument settings. Test samples prepared in the same buffer and combined with the same matrix are then used for analyses under the optimum instrument settings.

### Example 9

### Biomarker Detection

Cervical mucus is collected from women enrolled as part of an ongoing study of cervical neoplasia (14) as in Example 1. Protein samples are prepared using 6 samples from sponges with no visual blood contamination from women diagnosed with high-grade squamous intraepithelial lesion (HSIL) confirmed by colposcopy and/or biopsy (test samples) and women as a test group and 6 samples from women presenting negative Pap test and no prior history of abnormal cytology as a control group. Protein is extracted using M-PER® buffer (Thermo Fisher Scientific, Rockford, Ill.) containing 1× protease inhibitor (Roche, Indianapolis, Ind.). Total protein content is measured using the Coomassie Plus™ kit (Thermo Fisher Scientific) as per the manufacturer's protocol. The extracts are aliquoted and stored at −80° C. until assayed.

Each of the protein extracts are analyzed by SELDI using the protocol of Example 2. Each sample is spotted three times on the NP-20 sample plate and incubated for 1 h at room temperature (24° C.±2) and washed three times at 5 min intervals with the CM10 low stringency binding buffer, followed by a final wash with ddH2O. Chips are air-dried 30 min prior to the application of SPA matrix. The chips are analyzed on the SELDI-TOF instrument within 4 h of application of the matrix.

Data are collected using the instrument settings of Example 2. Each spectrum is individually analyzed as per Example 3. The detector response curves are evaluated using data from regions of the spectra interdispersed between visually identifiable peaks. Each of the mass data sets from each ionization is well described by Eq. 1. The values for each of the parameters are fit by least-squares analysis of each data set. The resulting quadratic variance functions are then used for quadratic variance preprocessing to create preprocessed data for each spectra as described in Example 4 and peaks are identified and matched as in Example 6.

The test samples identify several proteins with different abundances (intensities) relative to control samples. These proteins are identified as members of the ovalbumin serine proteinase inhibitors, cysteine proteinase inhibitors, and proteins involved in cellular glycolysis, cytokinesis, and metastasis. These results are in agreement with the proteins identified by an independent research group using traditional analyses (See Lema, C., et al., Proc Amer Assoc Cancer Res, Volume 47, 2006, Abstract #4455), but are reached much faster and with greater confidence that is achievable by prior methods.

### REFERENCES

1. McLerran D, Grizzle W E, Feng Z, Thompson I M, Bigbee W L, Cazares L H et al. SELDI-TOF MS whole serum proteomic profiling with IMAC surface does not reliably detect prostate cancer. Clin Chem 2008; 54:53-60.
2. Semmes O J, Feng Z, Adam B L, Banez L L, Bigbee W L, Campos D et al. Evaluation of serum protein profiling by surface-enhanced laser desorption/ionization time-of-flight mass spectrometry for the detection of prostate cancer: I. Assessment of platform reproducibility. Clin Chem 2005; 51:102-12.
3. Timms J F, rslan-Low E, Gentry-Maharaj A, Luo Z, T'Jampens D, Podust V N et al. Preanalytic influence of sample handling on SELDI-TOF serum protein profiles. Clin Chem 2007; 53:645-56.
4. McLerran D, Grizzle W E, Feng Z, Bigbee W L, Banez L L, Cazares L H et al. Analytical validation of serum proteomic profiling for diagnosis of prostate cancer: sources of sample bias. Clin Chem 2008; 54:44-52.
5. Cruz-Marcelo A, Guerra R, Vannucci M, Li Y, Lau C C, Man T K. Comparison of algorithms for pre-processing of SELDI-TOF mass spectrometry data. Bioinformatics 2008; 24:2129-36.
6. Emanuele V A, Gurbaxani B M. Benchmarking currently available SELDI-TOF MS preprocessing techniques. Proteomics 2009; 9:1754-62.
7. Meuleman W, Engwegen J Y, Gast M C, Beijnen J H, Reinders M J, Wessels L F. Comparison of normalisation methods for surface-enhanced laser desorption and ionisation (SELDI) time-of-flight (TOF) mass spectrometry data. BMC Bioinformatics 2008; 9:88.
8. Wegdam W, Moerland P D, Buist M R, Ver Loren van T E, Bleijlevens B, Hoefsloot H C et al. Classification-based comparison of pre-processing methods for interpretation of mass spectrometry generated clinical datasets. Proteome Sci 2009; 7:19.
9. Wei, W., Martin, A., Johnson, P. J., and Ward, D. G. 10 Years of SELDI: What Have we Learnt? Current Proteomics 7[1], 15-25. 2010.
10. Skold M, Ryden T, Samuelsson V, Bratt C, Ekblad L, Olsson H, Baldetorp B. Regression analysis and modelling of data acquisition for SELDI-TOF mass spectrometry. Bioinformatics 2007; 23:1401-9.
11. Malyarenko D l, Cooke W E, Adam B L, Malik G, Chen H, Tracy E R et al. Enhancement of sensitivity and resolution of surface-enhanced laser desorption/ionization time-of-flight mass spectrometric records for serum peptides using time-series analysis techniques. Clin Chem 2005; 51:65-74.
12. Emanuele, V. A. and Gurbaxani, B. M. Quadratic Variance Models for Adaptively Preprocessing SELDI Mass Spectrometry Data. BMC Bioinformatics. 2010; 11: 512.
13. Fung E T, Enderwick C. ProteinChip clinical proteomics: computational challenges and solutions. Biotechniques 2002; Suppl:34-1.
14. Rajeevan M S, Swan D C, Nisenbaum R, Lee D R, Vernon S D, Ruffin M T et al. Epidemiologic and viral factors associated with cervical neoplasia in HPV-16-positive women. Int J Cancer 2005; 115:114-20.
15. Panicker G, Ye Y, Wang D, Unger E R. Characterization of the Human Cervical Mucous Proteome. Clin Proteomics 2010; 6:18-28.
16. Panicker G, Lee D R, Unger E R. Optimization of SELDI-TOF protein profiling for analysis of cervical mucous. J Proteomics 2009; 71:637-46.
17. Andersch-Bjorkman Y, Thomsson K A, Holmen Larsson J M, Ekerhovd E, Hansson G C. Large scale identification of proteins, mucins, and their O-glycosylation in the endocervical mucus during the menstrual cycle. Mol Cell Proteomics 2007; 6:708-16.
18. Dasari S, Pereira L, Reddy A P, Michaels J E, Lu X, Jacob T et al. Comprehensive proteomic analysis of human cervical-vaginal fluid. J Proteome Res 2007; 6:1258-68.
19. Pereira L, Reddy A P, Jacob T, Thomas A, Schneider K A, Dasari S et al. Identification of novel protein biomarkers of preterm birth in human cervical-vaginal fluid. J Proteome Res 2007; 6:1269-76.
20. Shaw J L, Smith C R, Diamandis E P. Proteomic analysis of human cervico-vaginal fluid. J Proteome Res 2007; 6:2859-65.
21. Tang L J, De S F, Odreman F, Venge P, Piva C, Guaschino S, Garcia R C. Proteomic analysis of human cervical-vaginal fluids. J Proteome Res 2007; 6:2874-83.
22. Morris J S, Coombes K R, Koomen J, Baggerly K A, Kobayashi R. Feature extraction and quantification for mass spectrometry in biomedical applications using the mean spectrum. Bioinformatics. 2005; 21(9):1764-1775. doi: 10.1093/bioinformatics/bti254.
23. Fritsch F N, Carlson R E. Monotone Piecewise Cubic Interpolation. SIAM j Numerical Analysis. 1980; 17:238-246. doi: 10.1137/0717021.
24. Gould, W R, et al., J Biol Chem, 2004; 279(4):2383-93

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication is specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process for identifying optimal instrument detection parameters for a SELDI or MALDI mass spectrometer comprising:
subjecting a sample to SELDI or MALDI mass spectrometry to produce a first mass data set;
performing a fit of at least a portion of said first data set to a quadratic variance model to obtain a first quadratic variance function;
obtaining a first coefficient of variation function from said first quadratic variance function; and
identifying a first objective function in said coefficient of variation function.

2. The process of claim 1 further comprising
adjusting an instrument setting;
subjecting a sample to said mass spectrometry to produce a second mass data set;
performing a fit of at least a portion of said second data set to a quadratic variance model to obtain a second quadratic variance function;
obtaining a second coefficient of variation function from said second quadratic variance function;
identifying a second objective function in said coefficient of variation function; and
determining a minimum of said first objective function and said second objective function, wherein the instrument detection parameters used at said minimum represent optimized instrument detection parameters.

3. The process of claim 2 further comprising:
repeating the process of claim 1 a plurality of times.

4. The process of claim 1 further comprising obtaining a mass spectrum from said first sample.

5. The process of claim 2 further comprising adjusting mass spectrometer detection settings to said optimized detection parameters, and subjecting said sample or a second sample to MALDI or SELDI mass spectrometry using said optimized detection parameters.

6. The process of claim 1 wherein said portion of said data set is data between sample peaks within said data set.

7. The process of claim 1 wherein said sample is a buffer control sample.

8. (canceled)

9. (canceled)

10. The process of claim 1 wherein said quadratic variance functions have a variance that is constant for a peak with a mean intensity below 3700 and is quadratic for peaks with the mean intensity of 3,700 and 12,000.

11. The process of claim 1 wherein said quadratic variance function has a variance that is constant for a peak with a mean intensity above 12,000.

12. (canceled)

13. The process of claim 5 wherein said first sample or said second sample are proteinaceous.

14. (canceled)

15. The process of claim 4 wherein said spectrum includes 100 to 200 peaks with said spectrum in the range of 3 kDa-30 kDa for a proteinaceous sample.

16. The process of claim 1 wherein said data set includes 100 to 200 peaks in the range of 3 kDa-30 kDa for a proteinaceous sample.

17. A process for performing SELDI or MALDI comprising:
subjecting a sample to SELDI or MALDI mass spectrometry;
obtaining a mass spectrum comprising detection data from said sample;
subjecting said data to quadratic variance preprocessing to create preprocessed data; and
generating a preprocessed mass spectrum from said step of subjecting.

18. The process of claim 17 wherein the preprocessed data has a variance that is constant for a peak with a mean intensity below 3,700 and quadratic for the peak with the mean intensity of 3,700 and 12,000.

19. (canceled)

20. The process of claim 17 wherein said data for intensity peaks in the data for 2.5 to 30kDa by centroid mass.

21. The process of claim 17 wherein said spectrum includes 100 to 200 peaks with said spectrum in the range of 3 kDa -30 kDa for a proteinaceous sample.

22. A process for identifying the presence or absence of a biomarker in a sample comprising:
subjecting a sample to SELDI or MALDI mass spectrometry;
obtaining a mass data set comprising detection data from said sample;
subjecting said data set to quadratic variance preprocessing to create preprocessed data;
generating a preprocessed mass spectrum from said step of subjecting; and
identifying the presence or absence of a biomarker in said sample by analyzing said preprocessed mass spectrum for the presence or absence of a peak representing said biomarker.

23. The process of claim 22 wherein the preprocessed data has a variance that is constant for a peak with a mean intensity below 3,700 and quadratic for the peak with the mean intensity of 3,700 and 12,000.

24. (canceled)

25. The process of claim 22 wherein said data for intensity peaks in the data for 2.5 to 30kDa by centroid mass.

26. The process of claim 22 wherein said spectrum includes 100 to 200 peaks with said spectrum in the range of 3 kDa-30 kDa for a proteinaceous sample.
